(19) United States
(12) Patent Application Publication
(10) Pub. No.: US 2018/0249954 A1
(43) Pub. Date: Sep. 6, 2018

(54) SYSTEM AND METHOD FOR SIMULTANEOUS ELECTRICAL STIMULATION AND RECORDING FOR LOCATING NERVES DURING SURGERY

(71) Applicant: The Charles Stark Draper Laboratory, Inc., Cambridge, MA (US)

(72) Inventors: Daniel K. Freeman, Reading, MA (US); Andrew A. Berlin, Lexington, MA (US); Jesse Wheeler, Revere, MA (US)

(21) Appl. No.: 15/909,364

(22) Filed: Mar. 1, 2018

Related U.S. Application Data
(60) Provisional application No. 62/466,345, filed on Mar. 2, 2017.

(51) Int. Cl. A61B 5/00; A61B 5/04; A61B 17/3201

(52) U.S. Cl. CPC A61B 5/4893; A61B 5/04001; A61B 5/7405; A61B 2017/00893; A61B 5/7225; A61B 17/3201; A61B 2562/043; A61B 5/742

(57) ABSTRACT

This invention provides a system and method for performing a surgical procedure that effectively locates and allows a user to avoid engagement with hidden nerves in tissue in real-time. The system and method employs an integrated stimulating and sensing array. The array includes a plurality of spaced-apart electrodes, which are selectively stimulated while the electrodes then sense for a neural response. Each of the electrodes is stimulated to map of the sensed tissue region for localization of nerve paths. This localization can be stored and used to control cutting of tissue. The locations can be marked as nerve-free and/or no-go regions so as to avoid nerve-containing regions in subsequent procedures or following a stimulation procedure. This marking can be by any acceptable physical and/or virtual fiducial mechanism. The array can be a single structure with all relevant electrodes or some electrodes can be provided in a separate, remote probe assembly.

### RELATED APPLICATION

This application claims the benefit of co-pending U.S. Patent Application Ser. No. 62/466,345, entitled SYSTEM AND METHOD FOR SIMULTANEOUS ELECTRICAL STIMULATION AND RECORDING FOR LOCATING NERVES DURING SURGERY, filed Mar. 2, 2017, the teachings of which are expressly incorporated herein by reference.

### FIELD OF THE INVENTION

This invention relates to surgical procedures and more particularly to systems and methods for locating hidden anatomical features during surgery, such as nerves, and for controlling robotically assisted/automated surgical operations.

### BACKGROUND OF THE INVENTION

Unintended nerve damage during surgery causes chronic side-effects for millions of Americans each year. These injuries result in a wide range of effects, including neuropathic pain following back surgery, erectile dysfunction during prostate surgery, and reduced sensation of the lip and tongue following oral surgery. In each of these cases, the surgeon has a limited ability to see the nerves, which are often hidden from view and difficult to locate and track due to natural and stimulated motion within the tissue during surgery as well as the unpredictability of their locations in a particular patient's anatomy. Even employing the most advanced imaging system, the nerves tend to blend into the surrounding soft tissue.

Commercially available products and associated techniques for tracking nerves, such as the ProPep® Nerve Monitoring System by ProPep Surgical of Austin, Tex. and/or the NIM nerve monitoring system by Medtronic of Minneapolis, Minn., typically involve the use of electrical stimulation probes that excite the nerve at a specific location during surgery, together with EMG electrodes that detect the response of the muscle that the nerve innervates. Electromyography (EMG) is a diagnostic procedure typically employed to assess the health of muscles and the nerve cells that control them (motor neurons). Motor neurons transmit electrical signals under stimulation that cause muscles to contract. An EMG translates these signals into graphs, sounds or numerical values that a practitioner can interpret. In the current state of the art, stimulation of the nerve is performed at a single location, controlled by the surgeon using either a handheld probe or a robotically actuated probe. The resulting muscle stimulation is detected via EMG, with the EMG waveform displayed on a monitor in the operating room and in some cases accompanied by an audio or visual notification that a nerve has been detected at the present location of the probe.

These tools have several drawbacks, including the fact that the nerves are located in a separate step (or steps) from the actual cutting. In the process, the surgeon can lose track of their location, or they can move due to tissue deformation. Thus, nerve-related injuries continue to plague surgeons and patients. In addition, there may be instances in which neurons are excited by the electrical stimulus but the conventional EMG signal is generally too small to detect. As a result, the surgeon may be led to incorrectly believe that there are no nerves present because of the lack of an expected EMG response.

### SUMMARY OF THE INVENTION

This invention overcomes disadvantages of the prior art by providing a system and method for performing a surgical procedure that effectively locates and allows a user to avoid engagement with hidden nerves in tissue in real-time. The system and method employs an integrated stimulating and sensing array operating that generates an applicable response in nerves present in the tissue under analysis. The array includes a plurality of spaced-apart electrodes (e.g. microelectrodes), which are selectively caused to deliver electrical stimulation while other, or all, of the electrodes then sense the resulting neural response. Based on a cycle in which each of the electrodes is stimulated (e.g. by applying an appropriate level of electrical current for a predetermined duration), a map of the sensed tissue region can be constructed that allows for localization of any adjacent nerve paths. In general, a processor reads the response at each of the electrodes and can determine, based upon the timing (and amplitude) of a response voltage at each electrode the general location of the nerve, and its relative depth within the tissue adjacent to the array. This localization can be stored and used to control cutting of tissue (either manually or automatically) via feedback that enables the user/surgeon to avoid damaging nerves. In an embodiment, the locations can be marked as nerve free and/or no-go regions so as to avoid nerve-containing regions in subsequent procedures or following a stimulation procedure. This marking can be by any acceptable physical (e.g. surgical tattoo) and/or virtual (e.g. coordinates in stored image data) fiducial mechanism. The array can be a single structure with all relevant electrodes or some electrodes can be provided in a separate, remote probe assembly.

In an illustrative embodiment a system and method for locating nerve paths in tissue that can operate in real-time during a surgical procedure is provided. A surgical instrument tip is applied to the tissue at a region that can contain nerve paths. At this time the region is engaged with an array comprising a plurality of spaced-apart electrodes in conjunction with the instrument tip (i.e. the array is integral with the tip and moves with it through the tissue). The array is operated to apply electrical stimulating current to at least one of the electrodes, and a response to the stimulating current is received at (at least) some of the electrodes. Based on the stimulation and associated response, and with knowledge of the location of the various electrodes in the array with respect to a coordinate system, one or more nerve paths can be localized. Illustratively, each of the electrodes can be operated to apply the stimulating current in a step of an overall operational cycle. The receipt of the response to stimulation includes detecting the electrical response to the stimulating current at all of the electrodes in the array at a predetermined time after applying the stimulating current. The receipt can also include amplifying the response from each of the electrodes and filtering artifacts from the response. The response can cause generation of a feedback event (i.e. detecting presence of a nerve path), such as providing a lockout to operation of the instrument tip, sounding an alert, displaying a nerve location and/or applying a fiducial to the tissue in the region of the nerve path. In various embodiments, the instrument tip can comprise a manually operated or automated scissor. As a further step, the user can administer a muscle-contraction inhibiting drug to the tissue prior to operating the instrument or array so that motion within the tissue (e.g. muscle) is minimized. The array can overcome disadvantages of an EMG approach in which a minimized muscle response may not be detectable, while a direct nerve response can be detected by the array.

In another embodiment, a method for performing surgery on a living organism is provided. The exemplary surgery can be invasive, minimally invasive or, or substantially non-invasive. One step of the method includes locating nerve paths in tissue of the organism by, (a) applying an instrument tip to the tissue at a region that can contain nerve paths, (b) engaging the region with an array comprising a plurality of spaced-apart electrodes in conjunction with the instrument tip, (c) operating the array to apply electrical stimulating current to at least one of the electrodes and receiving a response to the stimulating current at at least some of the electrodes, and (d) determining localization of one or more nerve paths based on the response. Another step of the method includes guiding a surgical instrument (comprising the instrument tip and/or another instrument) through the tissue in a manner that takes into account the nerve paths.

### BRIEF DESCRIPTION OF THE DRAWINGS

The invention description below refers to the accompanying drawings, of which:

FIG. 1 is a diagram of an exemplary surgical arrangement including a surgical instrument fitted with an integral nerve stimulation and sensing array and associated controller and processor, according to an illustrative embodiment;

FIG. 2 is a diagram of the surgical instrument of FIG. 1 showing the illustrative nerve stimulation and sensing array, and associated assembly, in further detail;

FIG. 3 is a diagram showing a more detailed view of the array of FIG. 2; and

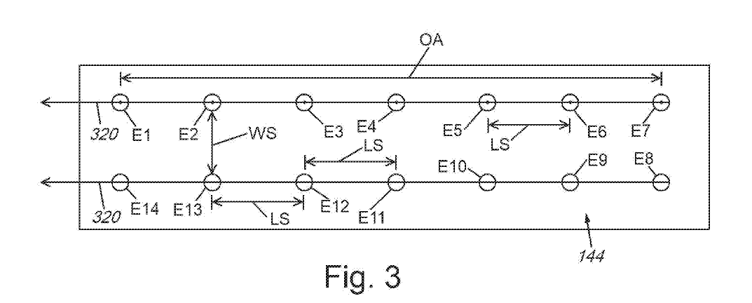

FIG. 4 is a diagram showing a flow diagram of an exemplary stimulation, sensing and nerve-localization procedure employing the array of FIGS. 1-3, according to an illustrative embodiment.

### DETAILED DESCRIPTION

I. System Overview

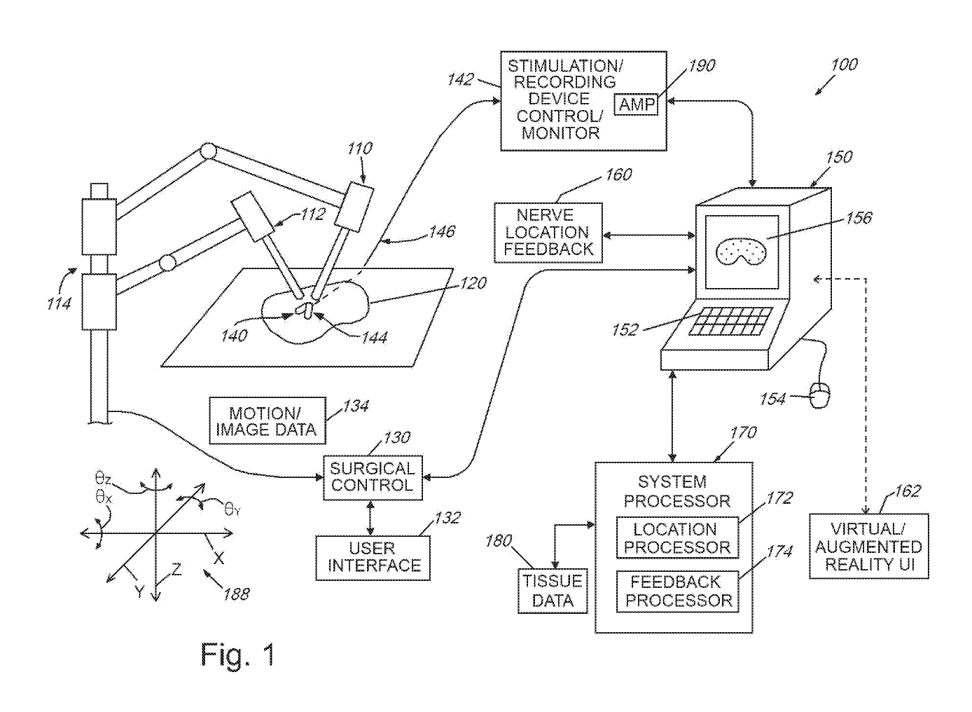

Reference is made to FIG. 1 which shows an exemplary surgical arrangement 100, that facilitates an associated surgical procedure. The arrangement and procedure, in this non-limiting example, is implemented by robotic (e.g. minimally invasive/laparoscopic) instruments 110, 112 that are mounted on a stand 114 that can be manually articulated or moved via robotic actuators and controllers along a plurality of degrees of freedom. In alternate arrangements, it is contemplated that surgery can be performed via open-cut or similar techniques using freehand-operated instruments. The instruments are shown inserted through incisions into a patient's body at an appropriate location/site (e.g. abdomen, groin, throat, head, etc.) 120, wherein they selectively engage one or more organs or other anatomical structures (e.g. muscles, vasculature, glands, ducts, etc.).

The instruments 110, 112 can include an appropriate video camera assembly with one or more image sensors that create a two-dimensional (2D) or three-dimensional (3D) still image(s) or moving image(s) of the site 120. The image sensors can include magnifying optics where appropriate and can focus upon the operational field of instrument's distally mounted tool(s). These tools can include forceps, scissors, cautery tips, scalpels, syringes, suction tips, etc., in a manner clear to those of skill. The control of the instruments, as well as a visual display can be provided by interface devices 130 and 132, respectively, based upon image/motion/location data 134 transmitted from the instruments 110, 112 and corresponding robotic components (if any).

Illustratively, at least one of the instruments includes a tip 140 (e.g. a cutting tip) with an integrated stimulation and sensing array 144 interconnected (by wired or wireless link 146 to a control and monitor 142. The control and monitor can be adapted from a commercially available or customized stimulation/recording electrophysiology device. It should be apparent to those of skill in the art that a variety of commercially available or custom-built electrophysiology amplifiers can be adapted for use with the present system and method. As described further below, the stimulation/recording device (and associated control/monitor components) 142 provides stimulus via the array at selected locations within the tissue, and measures the muscular response thereto.

By way of non-limiting example, the stimulation/recording device transmits data to a computing device 150, which can be implemented as a customized data processing device or as a general purpose computing device, such as a desktop PC, server, laptop, tablet, smartphone and/or networked “cloud” computing arrangement. The computing device 150 includes appropriate network and device interfaces (e.g. USB, Ethernet, WiFi, Bluetooth®, etc.) to support data acquisition from external devices, such as the stimulation/recording device 142 and surgical control/interface devices 130, 132. These network and data interfaces also support data transmission/receipt to/from external networks (e.g. the Internet) and devices, which can include various types of nerve location and nerve mapping feedback devices 160, as described below. The computing device 150 can include various user interface (UI) components, such as a keyboard 152, mouse 154 and/or display/touchscreen 156 that can be implemented in a manner clear to those of skill. The computing device 150 can also be arranged to interface with, and/or control, visualization devices, such as virtual reality (VR) and augmented reality (AR) user interfaces (UIs) 162. These devices can be used to assist in visualizing and/or guiding a surgeon (e.g. in real-time) using overlays of nerve structures on an actual or synthetic image of the tissue/organ being operated upon.

The computing device 150 includes a processor 170 according to embodiments herein, which can be implemented in hardware, software, or a combination thereof. The processor 170 receives data from the stimulation/recording device(s) 142 and from the surgical control and interface 130, 132 devices, and uses this information, in combination with additional data 180 (that can include stored information on nerve paths in the subject tissue/organ). A location process(or) 172 within the system processor 170 uses the information to determine relative locations of nerves bases upon the response provided by the array. This location information can be used to map the region, based upon the known location of the instrument tip 140 and associated array 144 relative to the tissue. The known instrument/array location can be characterized by a local instrument coordinate system and/or a global coordinate system 188 as shown (with nerve locations also being transformed into this global coordinate system). Note that the ability to localize and map nerves within tissue is further described in commonly assigned U.S. patent application Ser. No. 15/909,282, entitled SYSTEMS AND METHODS FOR SURGICAL TRACKING AND VISUALIZATION OF HIDDEN ANATOMICAL FEATURES, filed on Mar. 1, 2018, by Andrew A. Berlin, et al., the teachings of which are expressly incorporated herein by reference as useful background information. The system processor also includes a feedback process(or) 174, which interprets the location data generated by the location process(or) 172 and determines appropriate actions when the instrument location (received from the control 130) is proximate to a nerve.

II. Instrument Stimulating and Sensing Array

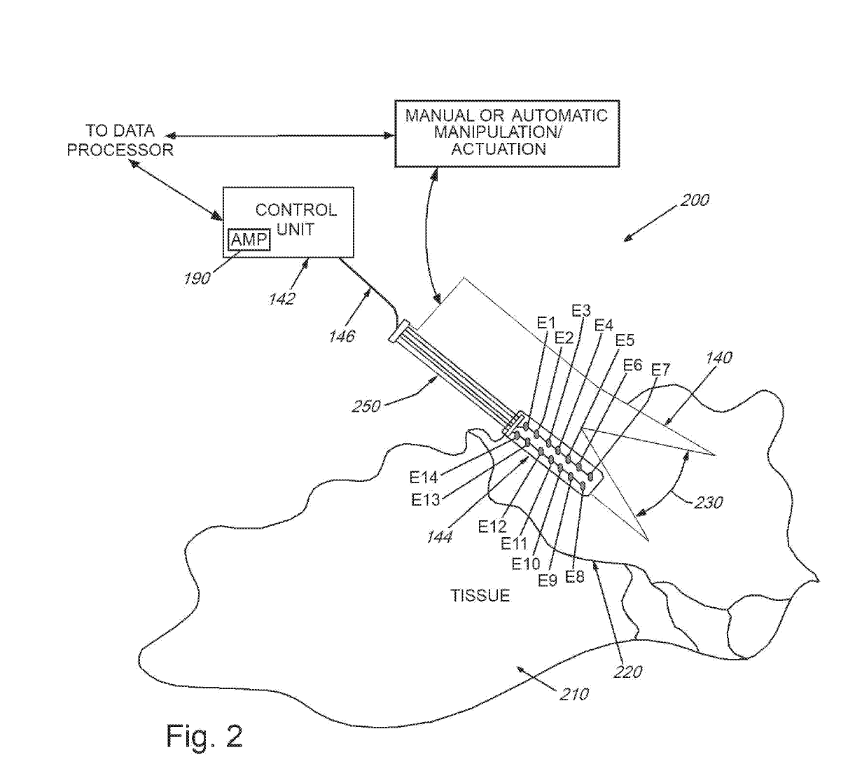

Reference is made to FIG. 2, which shows the above-described instrument tip (e.g. a surgical scissor/cutter) 140 that can be handheld, mounted at the end of a laparoscopic instrument or part of an automated surgical arrangement. The tip 140 includes a stimulation and sensing array 144 that consists of a plurality of individual microelectrodes E1-EN (which in this example, comprises a number, N=14, but is highly variable in alternate embodiments). The layout and spacing of electrodes is also highly variable, and can be based on the characteristics of the tissue being operated upon. By way of example, the scissor tip 140 is shown engaged with, and cutting into (double-curved arrow 230), a tissue region 210 that includes an exemplary nerve path 220. The objective of the embodiment is to ensure that the scissor tip 140 avoids this (and any other adjacent) nerve path, so as to minimize potential, permanent nerve damage.

The array 144 can be integral with (permanently mounted or removable) the scissor tip, as shown; and can be oriented with respect to the blades (or other tool tip structures, such as graspers, an electrocautery tip, or a scalpel blade) in a manner that allows the array 144 to engage the surface of the tissue with sufficient contact to enable transmission and receipt of electrical current though some or all of the electrodes. In embodiments, the array can be mounted on a flexible membrane so as to flex and conform to the shape of the underlying tissue. It can be otherwise mounted to springably flex and engage the tissue as the angle of attach of the scissor tip changes. In alternate embodiments some or all of the electrodes can be attached to a separate probe that is used in conjunction with the instrument tip and paced at a desired location with respect to the cutting blades. The movement of both the scissor and the probe can be tied together mechanically by appropriate mechanisms.

The electrodes E1-E14 are electrically connected to a lead assembly 250 of any acceptable arrangement. The leads interconnect to the control 142 via an appropriate link 146. As described below, the control 142 can include an amplifier circuit 190 that operates to amplify and resolve individual electrode signals. As shown, the electrodes E1-E14 in the exemplary array are mounted at a spacing (LS) from each other in the lengthwise direction and at a spacing (WS) in the widthwise direction. This spacing can be any acceptable value—for example, between 0.5 and 5 millimeters. As noted, the electrode spacing can vary, as well as the layout of electrodes (in this example, it defines a rectangle with two rows of seven), to accommodate the density of nerves in the subject tissue in the surgical field as well as the proposed cutting pattern. In alternate embodiments one row or three-or-more rows can be provided, or an alternate layout—e.g. concentric circles of electrodes—can be employed.

In general, each electrode can be adapted to act as both a stimulating and a recording electrode at the appropriate time in the cycle. The stimulation current can be made to return through one or more reference or ground electrodes simultaneously to control the direction and pattern of current flow in manner clear to those skilled in the art. As described below, the control causes each electrode to stimulate the tissue and the response is amplified and recorded by the other electrodes. This response is used to determine proximity of each electrode to a nerve path. Alternatively, the return and ground electrodes can be on one or more separate structure(s) located remote from the electrode array. Because there are limits as to how much current can be delivered through an electrode, particularly for microelectrodes, one possibility is to use an array of two-sizes of electrodes; the larger size (e.g. 0.5 mm diameter) can be used primarily for stimulation and for detecting large-scale neural activity such as compound action potentials. Conversely, the smaller size electrodes (e.g. 25 um diameter) can be used primarily for recording, allowing finer spatial precision and more electrodes to be fit in a given area.

The electrodes (E1-E14) can be constructed from any acceptable conductive material—for example, platinum, gold, silver, copper alloy, etc. that can be attached by (e.g.) soldering to underlying leads 320, or formed using (e.g.) vapor deposition, photolithography, or similar techniques.

III. Stimulation, Sensing and Localization Procedure

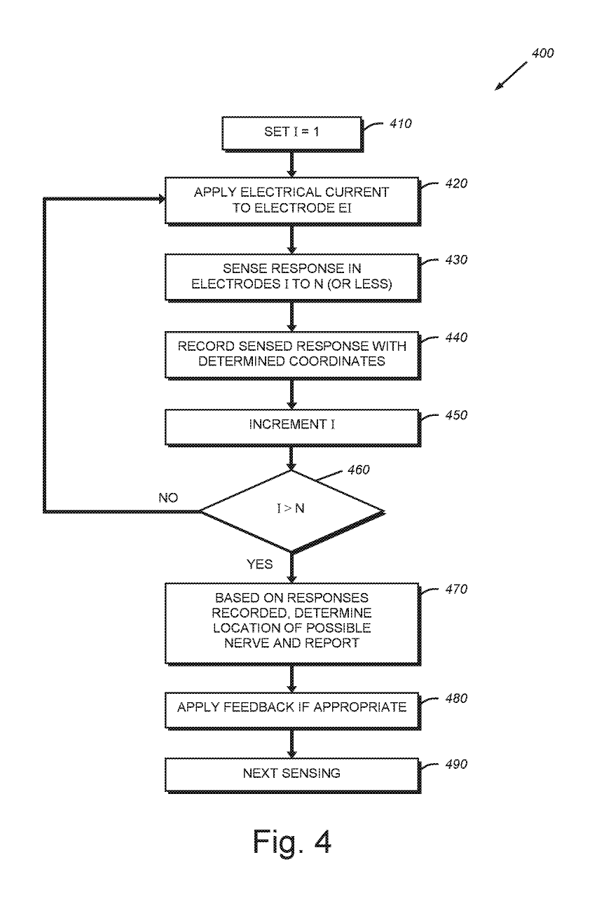

The above-described array 144 enables a novel technique for electrical stimulation and recording from nerves with an electrode array that is engaged against the tissue under examination. Reference is made to FIG. 4, which shows a procedure 400 for operating the array and employing results of the operation to localize nerve paths and generate feedback that can be used by the user (surgeon) or an automated system to perform certain tasks—for example, avoid cutting or relocate the scissor tip to avoid a nerve in the tissue. As shown the procedure 400 initiates a sense operation by setting an increment I equal to 1 (step 410). Note that this is an arbitrary step used to illustrate the array's sensing cycle. The electrode EI is stimulated so as to apply an electrical current through that electrode into the tissue in step 420. In step 430, the response from all electrodes (E1-EN) is sensed by the control, and in step 440, it is recorded (e.g. via the processor 170). This sensing can include the stimulating electrode, and determines whether there was any neural response induced as a result of the electrical stimulation. The recording process can include associating a position with respect to the tissue. The position can be determined by a variety of techniques. In general, the system recognizes the relative spacing of each electrode within the array and can se this to localize a nerve (for example, using advanced triangulation techniques). Note that fewer than all electrodes can be sensed in alternate embodiment—possibly where some electrode may insufficient contact with the tissue. In step 450, the value for I is incremented by (e.g.) 1 and the procedure 400 determines (in decision step 460), whether I has exceeded the maximum number of electrodes N. If I has not exceeded the N, then the procedure loops back to step 420 and the current is applied to the next, incremented electrode. Sensing, recoding and incrementing steps 430, 440 and 450, respectively repeat.

When the value for I has exceeded N, the procedure 400 branches to step 470 (via decision step 460), and determines the location of any potential nerve. Location procedures that employ various algorithms are employed. As an example of such an algorithm, the software in the processor 170 examines neural response at the recorded waveforms over a short pre-defined window following stimulation to determine whether the sensed voltage exceeds a predetermined threshold, including the possible presence of a compound action potential within adjacent nerves. Repeated stimulation and recordings allow the response to be averaged, thereby increasing the signal to noise (SNR) ratio. This allows the algorithm to report back to the user/surgeon that there is a nerve present in the area nearby the stimulating electrode (and hence the instrument tip). Repeating the stimulation (step 490) with multiple electrodes and/or at multiple probe locations generates a more complete map of the locations where nerves are present. This map can be stored and is presented to the user/surgeon in the form of appropriate feedback (step 480). This presentation can be visual (typically in conjunction with a surgical location tracking/navigation system and associated display (e.g. display 156), audible, tactile or thermal feedback (for example vibrating, heating or cooling the handle of the instrument or of a wearable bracelet), or other sensory feedback. The user/surgeon can then employ the information on the location of the nerve paths during the surgery to avoid cutting or damaging such nerves.

Referring again to FIGS. 1 and 2, an amplifier circuit 190 is provided in line with the array 144. This amplifier is adapted to allow the system to tolerate significant stimulus artifacts, as may be present in such a stimulation and sensing procedure. It can be constructed in accordance with skill in the art to provide appropriate amplification of the signal from each sensing electrode in the array—for example using multiple channels. More particularly, in a typical experiment involving electrical stimulation of neurons, an electrical current through one electrode can produce a voltage to appear on any nearby electrodes, and this is often referred to as a stimulus artifact. If the recorded voltage that results from this artifact exceeds the power supply voltage of the amplifier, or the operating range of the amplifier 190, then this can cause the amplifier to become saturated and consequently require tens or hundreds of milliseconds to recover. This means that it is often difficult to electrically stimulate tissue and then record from a nearby electrode (e.g. within several millimeters) because the amplifier will be saturated. Notably, the system avoids this potential saturation effect by using a custom amplifier that has stimulus artifact rejection capability built into its circuitry. The construction of such a rejection circuit should be clear to those of skill. In operation, the amplifier temporarily shuts down amplification during stimulation, and goes into sleep mode, thereby preventing saturation of the amplifier input. Once the stimulation is complete, amplification is then resumed and the system can record within approximately 0.1-1 ms temporal offset from the stimulus, allowing action potentials from nearby neurons to be detected. Because neuron action potentials can propagate at speeds that exceed 100 m/s in myelinated axons, it is desirable to position the recording electrodes far enough from the stimulating electrodes to avoid having the action potentials pass by while the amplifier 190 is still recovering. Notably, neurons that are excited with an electrical stimulus fire an action potential with a latency of 0.5-1ms, thereby allowing sufficient time for the amplifier to recover before action potential propagation begins. This phenomenon ensures that the amplifier can function adequately.

More generally, it should be noted that the location of a nerve can be determined by a combination of spacing and timing as measured by the array of electrodes. That is, the relative timing of the voltages across the electrode array can be used in a variety of ways to infer information related to nerve geometry and/or location within the tissue. In other words, the location processor can operate an algorithm that is based on the measurement of arrival time of the induced electrical pulse from one or more electrodes at a plurality of locations and time points. The algorithm thereby infers the location of the excitation based on the propagation velocity of the nerve impulse. Similarly, the effective velocity of propagation can be measured as the impulse traverses the array of electrodes, thereby indicating the likely depth of transition of the nerve. That is, a nerve that propagates into the tissue, as opposed to laterally on its surface, will have a time-space profile that involves decay of the signal over time at a more localized spatial coordinate than would a signal that is propagating laterally (which would typically have a comparable amplitude as it arrives at each spatial location).

A further challenge in detecting nerves is that stimulating the nerve often stimulates muscle contraction, which in turn, causes deformation/motion of the tissue in the area of interest. In an alternate embodiment, when a nerve is detected, in addition to (or instead of) notifying the user/surgeon, the system feedback activates an interlock that prevent the surgical instrument (which is manually or automatically controlled) from cutting tissue if a nerve is detected in its vicinity, or if a nerve has previously been detected in its vicinity.

In an embodiment, the system can include a marking mechanism (not shown) that is integrated with the instrument and/or array. Where tissue may be subject to deformation and/or motion a fiducial mark is placed on the tissue if a nerve is detected in the vicinity. For example, a surgical tattoo can be applied to the area using an appropriate device, via laser marking, thermal marking, application of a dye or other marking chemical, or other mechanism that creates a mark that can be detected optically, electrically, or mechanically. This fiducial mark can then be employed to automatically disable operation of cutting tools in the vicinity of the nerve, without the need to excite the nerve during the cutting operation itself. This retains the benefits of a stable (non-moving/deforming) region of tissue during a cutting procedure (i.e. there is no concurrent no nerve excitation, and hence potential induction of muscle contraction), while also providing the ability to preserve (avoid cutting/damaging) nerves. In a further alternate embodiment, tissue regions where it is safe to cut (nerve-free/safe-to-cut) are marked by the mechanism—for example at selected boundaries and/or predetermined increments along the surgical field—instead of marking the tissue regions that contain nerves (no-go).

It is expressly contemplated in alternate embodiments that the above-described marking can be virtual rather than physical. Algorithms, such as ‘optical flow’ can be used to track the location on the tissue where a nerve is detected. For example, using the texture properties of the tissue itself in conjunction with machine vision (pattern recognition) processes, the localized color pattern of the tissue, or other inherent physical properties such as position relative to key landmarks, the system can identify and track a specific location on the tissue without (free-of) the need to make a new permanent mark. In other words, use the marks/features that are already present for identification/tracking of location, while incorporating information captured by electrical detection probe into a location-oriented database that associates presence/absence of a nerve with location in the surgical field. A further description of a virtual localization and mapping of nerve paths is described in the above-incorporated U.S. Provisional Application Ser. No. 62/466,339.

IV. Operational Application

As described above, one of the challenges in performing certain surgeries, such as radical prostatectomy, is avoiding injuries to nerves. To minimize the chances of nerve injury, in present practice nerves are excited via electrical stimulation, leading to muscle response that is detected via EMG or a similar modality. This muscle response physically moves the tissue throughout the surgical field, making it more difficult to track the nerve and potentially causing damage to sensitive, partially-dissected tissues. In an embodiment, the muscle response is first inhibited through administration of a locally-acting drug that inhibits muscle contractions—for example, Dantrolene, Succinylcholine, or other muscle relaxants or paralytic agents. However, once muscle contractions are reduced via the administration of the inhibiting drug, the EMG signal that is presently used to detect nerve response to stimulation is far weaker, if it exists at all. The significantly reduces the effectiveness of EMG. Thus, it is desirable to detect propagation of the stimulated nerve action potential directly, without reliance on the EMG signal produced by muscle contraction. The electrical detection techniques described herein, combined with administration of muscle contraction inhibiting drugs, are an effective way to achieve the benefits of nerve location identification (and hence protection against accidental surgical injury) without the costs/complications associated with inducing muscle contraction and consequence motion/application of forces to the delicate tissues.

V. Conclusion

It should be clear that the above-described system and method effectively allows for real-time localization of nerve paths in a manner that provides meaningful feedback so that a user/surgeon can avoid cutting or otherwise damaging these regions during a surgical procedure. This system and method lends itself to generation of feedback that can be used to operate various automated safety mechanisms, as well as alarms and displays.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments of the apparatus and method of the present invention, what has been described herein is merely illustrative of the application of the principles of the present invention. For example, also as used herein, various directional and orientational terms (and grammatical variations thereof) such as “vertical”, “horizontal”, “up”, “down”, “bottom”, “top”, “side”, “front”, “rear”, “left”, “right”, “forward”, “rearward”, and the like, are used only as relative conventions and not as absolute orientations with respect to a fixed coordinate system, such as the acting direction of gravity. Additionally, where the term “substantially” or “approximately” is employed with respect to a given measurement, value or characteristic, it refers to a quantity that is within a normal operating range to achieve desired results, but that includes some variability due to inherent inaccuracy and error within the allowed tolerances (e.g. 1-2%) of the system. Note also, as used herein the terms “process” and/or “processor” should be taken broadly to include a variety of electronic hardware and/or software based functions and components. Moreover, a depicted process or processor can be combined with other processes and/or processors or divided into various sub-processes or processors. Such sub-processes and/or sub-processors can be variously combined according to embodiments herein. Likewise, it is expressly contemplated that any function, process and/or processor here herein can be implemented using electronic hardware, software consisting of a non-transitory computer-readable medium of program instructions, or a combination of hardware and software. Accordingly, this description is meant to be taken only by way of example, and not to otherwise limit the scope of this invention.

What is claimed is:

1. A method for locating nerve paths in tissue comprising the steps of:
applying a surgical instrument tip to the tissue at a region that can contain nerve paths;
engaging the region with an array comprising a plurality of spaced-apart electrodes in conjunction with the instrument tip;
operating the array to apply electrical stimulating current to at least one of the electrodes and receiving a response to the stimulating current at at least some of the electrodes; and
determining localization of one or more nerve paths based on the response.

2. The method as set forth in claim 1, wherein the step of operating includes operating each of the electrodes to apply the stimulating current in a step of an overall operational cycle.

3. The method as set forth in claim 2, wherein the step of receiving includes detecting the electrical response to the stimulating current at all of the electrodes in the array at a predetermined time after applying the stimulating current.

4. The method as set forth in claim 3, further comprising amplifying the received response from each of the electrodes and filtering artifacts from the response.

5. The method as set forth in claim 4, further comprising generating a feedback event based upon the step of determining.

6. The method as set forth in claim 5, wherein the step of generating includes providing a lockout to operation of the instrument tip, sounding an alert, displaying a nerve location and applying a fiducial to the tissue.

7. The method as set forth in claim 1, wherein the instrument tip comprises a manually or automated scissor.

8. The method as set forth in claim 1, wherein the array is integral with the instrument tip.

9. The method as set forth in claim 1, further comprising administering a muscle-contraction inhibiting drug to the tissue.

10. The method a set forth in claim 1, wherein the step of determining localization includes determining at least one of a timing of the response and an amplitude of the response relative to application of the stimulating current.

11. A system for locating nerve paths in tissue:
an array comprising a plurality of spaced-apart electrodes that operate in conjunction with a surgical instrument tip;
a control that applies electrical stimulating current to at least one of the electrodes and receives a response to the stimulating current at at least some of the electrodes; and
a process that localizes one or more nerve paths based on the response.

12. The system as set forth in claim 11, wherein each of the electrodes is arranged to apply the stimulating current in a step of an overall operational cycle.

13. The system as set forth in claim 12, wherein the controller is arranged to receive the response to the stimulating current at all of the electrodes in the array at a predetermined time after applying the stimulating current.

14. The system as set forth in claim 13, further comprising an amplifier that amplifies the received response from each of the electrodes and filters artifacts from the response.

15. The system as set forth in claim 14, further comprising a process that generates a feedback event based upon localization of the one or more nerve paths.

16. The system as set forth in claim 15, wherein the feedback event comprises at least one of a lockout to operation of the instrument tip, sounding an alert, displaying a nerve location and applying a fiducial to the tissue.

17. The system as set forth in claim 11, wherein the instrument tip comprises a manually or automated scissor.

18. The system as set forth in claim 11, wherein the array is integral with the instrument tip.

19. The system as set forth in claim 11, wherein the process that localizes the nerve path is arranged to determine at least one of a timing of the response and an amplitude of the response relative to application of the stimulating current.

20. A method for performing surgery on a living organism comprising the steps of:
locating nerve paths in tissue of the organism by,
(a) applying an instrument tip to the tissue at a region that can contain nerve paths,
(b) engaging the region with an array comprising a plurality of spaced-apart electrodes in conjunction with the instrument tip,
(c) operating the array to apply electrical stimulating current to at least one of the electrodes and receiving a response to the stimulating current at at least some of the electrodes, and
(d) determining localization of one or more nerve paths based on the response; and
applying a surgical instrument to the tissue in a manner that takes into account the nerve paths.
